(19) United States
(12) Patent Application Publication
(10) Pub. No.: US 2017/0080127 A1
(43) Pub. Date: Mar. 23, 2017

(54) Biologically active graft for skin replacement therapy

(71) Applicant: Carlos A. Alvarado, Guatemala (GT)

(72) Inventor: Carlos A. Alvarado, Guatemala (GT)

(21) Appl. No.: 14/756,601

(22) Filed: Sep. 23, 2015

(51) Int. Cl. A61L 27/60; A61L 27/36; A61F 2/10; A61L 27/54

(52) U.S. Cl. CPC A61L 27/60; A61L 27/54; A61L 27/3604; A61F 2/105; A61L 2430/34; A61L 2300/412

(57) ABSTRACT

The present invention relates to a biologically active graft (BAG) composition and methods for its preparation and use.

More particularly, it details a method for preparing a non-immunogenic tissue graft composition comprising esophagus mucosa, basement membrane and tunica submucosa as intact natural sheet forms (Ad integrum), delaminated from the tunica muscularis and adventitia of an esophagus of a warm-blooded vertebrate.

The composition is capable of serving as a substitute for skin autograft for skin replacement therapy and induces stromal cells regeneration when implanted in warm-blooded vertebrate including human subjects.

### CROSS-REFERENCE TO RELATED APPLICATIONS

“Not Applicable”

### STATEMENT REGARDING FEDERALLY SPONSORED RESEARCH OR DEVELOPMENT

“Not Applicable”

### THE NAMES OF THE PARTIES TO A JOINT RESEARCH AGREEMENT

“Not Applicable”

### REFERENCE TO A “SEQUENCE LISTING

“Not Applicable”

### STATEMENT REGARDING PRIOR DISCLOSURES BY THE INVENTOR OR A JOINT INVENTOR

“Not Applicable”

### TECHNICAL FIELD

Field of the Invention

The present invention relates to a biologically active graft composition and methods for its preparation and use. More particularly, the present invention is intended for manufacture of non-immunogenic tissue graft compositions comprising esophagus mucosa, basement membrane and tunica submucosa of an esophagus of a warm-blooded vertebrate capable of being utilized as a substitute for skin autograft for skin replacement therapies and for inducing stromal cells regeneration when implanted into warm-blooded vertebrates including humans.

Description

A biologically active graft (BAG) composition comprising esophagus mucosa, basement membrane and tunica submucosa as intact natural sheet forms (Ad integrum), delaminated from the tunica muscularis and adventitia of a esophagus of a warm-blooded vertebrate, and therapeutic applications arising from their utilization is described.

This new BAG composition differs from previously known tissue derived from esophagus mucosal tissues of U.S. Pat. No. 5,300,306 by the inclusion or retention of a mainly intact basement membrane and connective tissue (tunica submucosa) in the present invention.

This connective tissue is critical to regenerating the in vivo milieu at the wound site, and the three-dimensional structure of the new construct acts as a substitute for skin autograft in skin replacement therapies and for inducing stromal cells regeneration.

The BAG composition adheres to connective tissues, promotes angiogenesis, and achieves permanent engraftment of Dermis and has been used for many years in humans without evidence of adverse immunological outcomes.

At the beginning of our study, several years ago, the goal was to find the absolutely optimal dermal substitute, an objective that is fulfilled by the present invention.

The BAG composition is capable of being utilized as a substitute for skin autograft in skin replacement therapies, and for inducing stromal cells regeneration with levels of performance and outcome very similar to the most sophisticated commercially available tissue-engineered product that is Integra® (Integra Life Sciences Corp., Plainsboro, N.J., USA).

### BACKGROUND ART

Background of the Invention

The Applicants are co-inventors of a Tissue-equivalent membrane from bovine esophageal tissue that is useful for the proliferation of epidermal cells in treating skin loss or injury. See U.S. Pat. No. 5,300,306

This new BAG composition differs from previously known tissue compositions derived from esophagus mucosal tissues, by the inclusion of a mainly intact basement membrane and connective tissue (tunica submucosa) in the present invention.

This connective tissue is critical to regenerating and maintaining the in vivo milieu at the wound site, and the three-dimensional structure of the new construct serves as a substitute for skin autograft in skin replacement therapies, and for inducing stromal cells regeneration.

In one non-limiting example, the BAG composition has physical and physiological properties similar to that of the native tissue architecture of the organ to be replaced/repaired, since there is an outer epidermal analog (non-keratinized stratified squamous epithelium), and inner dermal analog (basement membrane and connective tissue).

In this manner, a solid composition can be produced that has greater physical strength than is the case with an epithelial mucosa alone, consequently it is suitable for surgical handling to ensure a long-lasting barrier function.

For example and without limitation, where a patient has had tissue removed due to trauma, debridement and/or removal of damaged, diseased or cancerous tissue, or other surgical intervention, the composition can be utilized at the site of tissue removal to facilitate in-growth of connective tissue.

The BAG composition adheres to connective tissue, promotes angiogenesis, and achieves permanently engraftment of Dermis.

The BAG composition, a xenograft by nature, moves away from the typical performance and documented complication of these materials.

On the other hand, the BAG composition is capable of being utilized as a substitute for skin autograft in skin replacement therapies and for inducing stromal cells regeneration with levels of performance and outcome very similar to the most sophisticated commercially available tissue-engineered product that is INTEGRA® (Integra Life Sciences Corp., Plainsboro, N.J., USA).

The ability of complex substrates to support cell growth has been previously reported, and matrix products supporting such growth are commercially available. For example, U.S. Pat. No. 4,060,081 (Yannas et al, 1977), the teachings of which are hereby incorporated by reference, discloses a multilayer membrane suitable for use as synthetic skin commercially available at this time by the name of Integra®.

Integra® (Integra Life Sciences Corp., Plainsboro, N.J., USA) is a dermal regeneration template consisting of bovine collagen, chondroitin-6-sulphate and a Silastic membrane. This product has gained widespread use in the clinical treatment of deep partial-thickness and full-thickness burn wounds, full-thickness skin defects of different etiologies, chronic wounds and in soft tissue defects (Elliot et al, 1973) (Song et al, 1966) (Halim et al, 2010) (Van der Veen et al, 2010)(Hansen et al, 2001) (Burke J F, Yannas IV et al, 1981) (Heimbach et al, 1988). The bovine collagen dermal analogue integrates with the patient's own cells and the temporary epidermal silicone is peeled away as the dermis regenerates. A very thin autograft is then grafted onto the neo-dermis (Pham et al, 2007).

Also matrix products supporting cells growth include preparations derived from small intestinal submucosa or SIS now commercially available, include, but are not limited to, Oasis™, Surgisis™, Surgisis-ES™, Stratasis™, and Stratasis-ES™ (Cook Urological Inc.; Indianapolis, Ind.) and GraftPatch™ (Organogenesis Inc.; Canton Mass.) and the ECM derived from urinary bladder (UBM). Commercially available preparations include, but are not limited to UBM (Acell Corporation; Jessup, Md.).

Another approach to tissue repair consists of the treatment of tissue with a biodegradable polymeric material which, acting as a scaffold; include biodegradable polymeric material formed from crosslinked collagen molecules that are covalently bonded to glycosaminoglycan molecules.

These polymer materials have been described in the scientific literature and the patent literature. See, for example, U.S. Pat. No. 4,505,266 (Mar. 19, 1985); U.S. Pat. No. 4,448,718 (May 15, 1984); U.S. Pat. No. 4,418,691 (Dec. 6, 1983); U.S. Pat. No. 4,458,678 (Jul. 10, 1984); U.S. Pat. No. 4,350,629 (Sep. 21, 1982); U.S. Pat. No. 4,522,753 (Jun. 11, 1985); U.S. Pat. No. 4,280,954 (Jul. 28, 1981); U.S. Pat. No. 4,252,759 (Feb. 24, 1981); and U.S. Pat. No. 4,060,081 (Nov. 29, 1977) the teachings of which are incorporated herein by reference.

U.S. Pat. No. 6,893,666 discloses a composition and methods for using a tissue regenerative matrix to promote the restoration, remodeling or repair of connective tissue. The composition of the matrix comprises devitalized mammalian epithelial basement membrane of the intestine and tunica propria, which can further include submucosa, tunica muscularis, growth factors, a cell, or a polymer. For the purposes of this invention, devitalized means acellular or substantially acellular which differs in the present invention which preserves the cells in their natural form.

These other synthetic skin substitutes generally lack basement membrane and architecture that resemble human skin configuration but the material of this invention differs in this important aspect: the esophagus of warm-blooded vertebrate that retains its basement membrane in its native composition and in a histological point of view: is very similar to human skin.

The inventor notes that for every facet in this process, further research is still needed.

Current data in the form of large, randomized, controlled trials are lacking; however, the data that is available suggests that reliable, definitive, long-term repair of complicated burns-wound defects and other types of injury are achievable.

In conclusion it can be stated that much progress has been already been made in the past ten years, and that the results obtained thus far show great promise with regard to the future clinical application of the new biologically active graft (BAG) composition.

### BRIEF SUMMARY OF THE INVENTION

This invention provides a proven technology to produce a non-immunogenic tissue graft compositions comprising esophagus mucosa, basement membrane and tunica submucosa of an esophagus of a warm-blooded vertebrate. The composition can be utilized for implanting at the site of tissue removal for immediate wound closure in cases where native tissue is damaged, in one embodiment, by trauma, or in another embodiment, by burns, cancer, vascular skin complications (chronic ulcers) or extensive injuries presenting in theaters of war (full-thickness burn-wounds defects).

The applicant observed that the composition has physical and physiological properties similar to that of the native tissue architecture of the organ to be replaced/repaired, since in a histological point of view is very similar to human skin, thus becomes an considerable importance of the invention and the general research intensity in the field of skin replacement therapy.

The patent aims to protect the claims related to the process for producing the composition, and claims relating to the methods used to implant the composition by surgical techniques in human subjects.

### BRIEF DESCRIPTION OF THE SEVERAL VIEWS OF THE DRAWING

“Not applicable”

### DETAILED DESCRIPTION OF THE INVENTION

Definitions

In the discussions herein, a number of terms are used. In order to provide a clear and consistent understanding of the specification and claims, the following definitions are provided.

Allograft—Graft of tissue between individuals of the same species but of disparate genotype.

Antiseptic—A substance that inhibits the growth and development of microorganisms without necessarily killing them. Antiseptics are usually applied to body surfaces.

Autograft—Graft of tissue derived from another site in or on the body of the organism receiving it.

Basement membrane—Between the epidermis and dermis lies the basement membrane (basal lamina), which keeps the outer layer adhered closely to the lower layer, term usually used in light microscopy.

Biocide—A general term for any agent that kills organisms.

Biocompatible—Refers to compositions (e.g. cells, tissues, matrices, etc.) that do not substantially disrupt the normal biological functions of other compositions to which they contact

Connective tissue—Refers to tissue that protects and supports the body and its organs, and tissues that bind organs together.

Chemical germicide—A chemical or a mixture of chemicals used to kill microorganisms.

Decontamination—Any process for removing and/or killing microorganisms.

Dermal autograft—Skin autograft from which epidermis and subcutaneous fat have been removed; used instead of fascia in various plastic (surgery) procedures.

Debridement—Is the removal of loose, devitalized, necrotic, and/or contaminated tissue, foreign bodies, and other debris on the wound using mechanical or sharp techniques

Disinfectant—A chemical or mixture of chemicals used to kill microorganisms, usually applied to inanimate surfaces or objects.

Disinfection—A physical or chemical means of killing microorganisms, but not necessarily spores.

Dressing—Any of various materials utilized for covering and protecting a wound.

Engraftment—Incorporation of grafted tissue into the body of the host.

Epithelial tissue—Refers to the cellular (typically avascular) layer covering all the free surfaces, cutaneous, mucous, and serous, including the glands and other structures derived there from.

Excision—Is a surgical procedure requiring incision through the deep dermis (including subcutaneous and deeper tissues) of open wounds, burn eschar, or burn scars.

Graft—Any tissue or organ for implantation of transplantation.

Granulation tissue—Newly formed vascular tissue normally produced in the healing of wounds of soft tissue and ultimately forming the scar; it consists of small, translucent, red, nodular masses of granulations that have a velvety appearance.

Host—Refers to an individual (whether human or non-human) into whom cells, tissue, blood, or organs are transplanted.

Matrix—Refers to a composition comprising a plurality of pores dividing free space into partially enclosed interstices wherein said interstices are in fluidic communication

Microbicide—A chemical or mixture of chemicals that kills microorganisms.

Replacement—Is used only as an adjective in the context of ‘skin replacement surgery’

Sterilization—A process that kills and/or removes all classes of microorganisms and spores.

Substitute—Is used only as a noun in the context of ‘substitute for skin graft’.

Skin allograft—An allograft consisting of intact viable or non-viable epidermal and dermal tissue.

Skin autograft—An autograft consisting of intact viable epidermal and dermal tissue.

Skin Substitute—A biomaterial, engineered tissue, or combination of biomaterials and cells or tissues that can be substituted for a skin allograft, a skin autograft, an epidermal autograft, or a dermal autograft in a clinical procedure.

Stroma—The supportive framework of an organ usually composed of connective tissue.

Tissue regeneration—Healing in which lost tissue is replaced by proliferation of cells which reconstruct the normal architecture.

Tissue repair—Healing in which lost tissue is replaced by a fibrous scar, which is produced from granulation tissue.

Xenograft—A graft of tissue transplanted between animals of different species

The invention is directed to biologically active graft, methods of producing the same, and therapeutic applications arising from their utilization.

In one embodiment, the term ‘biologically active graft’, ‘(BAG)’ or ‘graft’ refers to three-dimensional structure for implantation or transplantation that induced stromal cells regeneration.

In another embodiment, any reference to ‘including humans’ placed after references to ‘warm blooded vertebrates’ is intended to clarify that whilst humans are contemplated as recipients of the BAG, human donors of such tissue are not contemplated, this clarification is provided because the term ‘warm blooded vertebrates’ is used both in the meaning of donor and recipient within this document.

In accordance with one embodiment, the esophagus of a warm-blooded vertebrate is used in the preparation of a biologically active graft (BAG) composition. Such tissue graft compositions enables a surgeon to utilize this composition in a diverse range of surgical applications such as burns, wounds, scalps, cancer removal, vascular skin complications (chronic ulcers) or extensive injuries presented in theaters of war.

The preparation of a (BAG) composition from the esophagus of warm-blooded vertebrate in accordance with the invention is carried out by separating the mucosa, basement membrane and tunica submucosa from the esophagus of warm-blooded vertebrate in a manner that ensures minimal disruption or damage to this natural arrangement.

Conservation of these components from the esophagus of warm-blooded vertebrates results in the preparation of a (BAG) composition that is non-immunogenic, and, thus, does not induce an adverse host immune response when it is implanted into a host.

The method comprises the steps of delaminating the mucosa, basement membrane and tunica submucosa from the whole esophagus of warm-blooded vertebrate.

Accordingly, one embodiment of this invention provides a method for preparing a natural cellular/nonliving composition comprising non-keratinized stratified squamous epithelium (Mucosa), basement membrane and connective tissue (tunica submucosa) in its natural sheet forms (Ad integrum) from the esophagus of a warm-blooded vertebrate.

In another embodiment the mucosa, basement membrane and tunica submucosa for use in the (BAG) composition in accordance with the invention are typically prepared from the esophagus of a warm-blooded vertebrate harvested from animals including, for example, pigs, cattle, sheep or other warm-blooded vertebrates and does not exclude the use of the esophagus of cold-blooded aquatic animals and other marine mammals.

In another embodiment, this invention provides a process for preparing a biocompatible graft composition from the esophagus of warm-blooded vertebrate, and in general, the method for preparing the mucosa, basement membrane and tunica submucosa comprises the steps of obtain freshly harvested esophagus of warm-blooded vertebrate from proper suppliers that ensure full traceability and in a First Process the material is washed in a large volume of circulating water to remove adhering blood and residues of food, followed by immersing in 0.1% (v/v) peracetic acid and 95.9% (v/v) sterile water for two hours.

Then, in a Second Process the operator proceeds to cut the material in the longitudinal axis using surgical instruments, and carefully proceed to separate the adventitia and muscular layers in order to preserve intact the inner structures (mucosa, basement membrane and tunica submucosa) because they are the primary material of the present invention. Then the Larger pieces of esophagus can be cut in segments 8 cm long×5 cm wide. Next, in each segment, the peracetic acid residue is removed by washing twice for 15 minutes with PBS (Buffered saline) (pH=7.4) and twice for 15 minutes with sterile water in order to remove any residual disinfectant-sterilizing agent. Each tissue should be packaged individually as soon as possible into a vacuum bag (multi-layer synthetic bag). Air is removed from the bag.

For preservation, the applicant can opt for placing the material in the freezer and this preserves it well until the time of its use, with a storage life of more than one year free of microorganisms (which has been confirmed by multiple cultivation of random samples). At this time, the product is preserved with lyophilization.

Thus, another embodiment of this invention is a method of implanting into the vertebrate the BAG composition, prepared as described above comprising mucosa, basement membrane and tunica submucosa of esophagus of warm-blooded vertebrate in an amount effective to induce cells regeneration of connective tissues at the site of administration of the graft.

The BAG composition produced and used in accordance with this invention, upon implantation, can serve as a template for supporting the growth of new endogenous connective tissue and for induced stromal cells regeneration in the warm-blooded vertebrate including humans.

Connective tissues for the purposes of the present invention include the dermal layer of skin. In accordance with this embodiment, the tissue graft compositions are used beneficially to induce the formation of stromal cells at a desired site in a warm-blooded vertebrate including humans.

In another embodiment, the inclusion of a mainly intact basement membrane and connective tissue (tunica submucosa) from the esophagus of warm-blooded vertebrate in the present invention, produce a template with more consistent physical properties and more acceptable clinical composition than the previous description by the applicant.

In another embodiment it is also expected that the BAG composition of the invention may contain additional bioactive components including, for example, one or more of glycosaminoglycans, glycoproteins, proteoglycans, and/or growth factors naturally found in tissues where the scaffolding is implanted for applications such as, for example, tissue repair or regeneration.

In another embodiment, this invention provides a method of implanting a graft of this invention in a subject for immediate wound closure and permanent regeneration of dermis, as show in example # 2 in this discovery.

In one embodiment, the size and form of said biologically active graft is a function of the tissue into which the graft is to be implanted.

In another embodiment, the biologically active graft is implanted with the mucous side facing outward and the remaining layer side facing inward in direct contact with the wound bed.

In one embodiment, the membrane becomes sufficiently adhered to the graft bed so that the graft becomes thoroughly integrated at all levels and will promote the regeneration of the dermis, induces angiogenesis from the surrounding connective tissue and can help prepare the wound area for skin autograft where such is considered additionally required.

In another embodiment, use of the graft for repair, regeneration of tissue is in cases where native tissue is damaged, in one embodiment, by trauma, or in another embodiment, by burns, cancer, vascular skin complications (chronic ulcers) or extensive injuries presenting in theaters of war.

In another embodiment, is contemplated the use of esophagus mucosa, basement membrane and tunica submucosa from the esophagus of a warm-blooded vertebrate for the manufacture of other treatment methods or medical applications or products which have not been discussed in the present application, e.g., topical formulations (creams, ointments, gels), transdermal systems or used for the purpose of improving, developing or enhancing other biotechnological/biological products.

As can be seen from the forgoing description, the concepts of the present disclosure provide numerous advantages

The following examples are presented in order to more fully illustrate the preferred embodiments of the invention. They should in no way be construed, however, as limiting the broad scope of the invention.

### EXAMPLE 1

### Mode for Invention

Method of Making the Biologically Active Graft composition

Description

The biologically active graft (BAG) composition is made from the oesophagus of warm blood vertebrates. The esophagus comprises distinct layers. From the inner to the outside, these are: mucosa, submucosa, muscle and adventitia. The mucosa and submucosa (include the basement membrane) forms the main foundation in the present invention.

Source:

The biologically active graft (BAG) composition is obtained from animals that have passed ante mortem and post mortem inspection and meet regulatory requirements for not poisonous products. Only materials from proper suppliers that ensure full traceability should be used. Manufacturers should provide a certificate of compliance for every lot of materials should take into account the quality of tissues to be removed and their appropriate handling after removal.

Process ONE:

Evisceration stages: At the site of animal sacrifice the oesophagus was separated from the trachea and lungs in order to tie it, which allowed the lungs to be removed through the diaphragm without rupturing the oesophagus. This is primarily a hand and/or knife operation. Both the inner and outer layers of the esophagus are carefully removed, and appropriate gloves must be wearing for all procedures.

After harvest the esophagus, should be inspected in relation to the smell, irregularity or abnormality in shape or color and discard the material if any such defect be present. Them the primary material is transfer torn an area that allows copious amounts of circulating fresh water for cleaning its external and internal surfaces and mainly run water through the lumen of the esophagus.

Next desirable processes of the invention involve immersing the tissue source (e. g. by submersing) in 0.1% (v/v) peracetic acid and 95.9% (v/v) sterile water for two hours. Laboratory.

Working Areas

The laboratory should be kept neat, clean and free of materials that are not pertinent to the work.

The surfaces of Stainless steel table should be decontaminated before and after each use and application of pressure with vigorous scrubbing greatly improves the removal of grease and other unwanted contaminants from a preparation surface. Freshly prepared hypochlorite solutions recommended for decontamination at 1 g/l.

All instruments used during the procedure should be sterile and stored on sterile drape.

Process TWO:

In the previously disinfected surface of Stainless steel table and covered with a sterile drape, the tissues may be removed from their container and laid upon table, using an aseptic technique with sterile cloths and gloves.

Using surgical scissors or scalpels proceed to cut the material completely in the longitudinal axis, to identify macroscopically the structures that make the composition. The mucosal layer is found inside the cylindrical tube which in the normal esophagus is recognized for having smooth surface and white color.

The material thus obtained was placed over the area to be processed with the plane mucous put downward. Using surgical instruments, such as Metzenbaum scissors or scalpel, carefully proceed to separate the adventitia and muscular layers (which is recognized by its red color) in order to preserve intact the inner structures because they are the primary material of the present invention.

The mucosa, basement membrane and tunica submucosa are separated easily and consistently from the whole esophagus and should be examined to confirm there is minimal tissue debris on its surface.

Larger pieces of esophagus tissues can be reduced in size and cut in segments 8 cm long×5 cm wide (this may vary slightly according to the animal that is used as source material).

Next, in each segment, the peracetic acid residue is removed by washing twice for 15 minutes with PBS (Buffered saline) (pH=7.4) and twice for 15 minutes with sterile water in order to remove any residual disinfectant-sterilizing agent.

Each tissue should be packaged individually as soon as possible packaging into a vacuum bag (multi-layer synthetic bag). Air is removed from the bag by means of the vacuum packaging machine and the bag then is sealed.

For preservation it is permitted to place the material in the freezer and keep well until the time of its use, this has been a preferred method, which has resulted in a stable material, free of microorganisms (which have been confirmed by multiple cultivation of random samples of the packaging material). Freezing and frozen storage can give a storage life of more than one year.

It is contemplated the use of other method for final sterilization, such as exposure to ultraviolet radiation, low temperature hydrogen peroxide sterilization, or gamma irradiation.

### EXAMPLE NO. 2

Biologically Active Graft (BAG) Placement Procedure

Preventive aspect: In elective surgery, patients should abstain from taking aspirin for 10-14 days before surgery; alcohol, vitamin E, and NSAIDs for 4-5 days before surgery; and Coumadin for 3-5 days before surgery which may contribute to postoperative hematoma or seroma formation. The (BAG) composition is not suitable for placement onto infected wounds.

Management of the recipient wound bed: this is probably the most important prerequisite for successful skin grafting of the BAG composition. In essence, in fresh wound beds (surgical wound or recent accidental wounds) where is possible, the recipient bed should be vascular and free of infection and devitalized or necrotic tissue, and in burn patient's careful excision or serial tangential debridement is perhaps the most important step prior to BAG composition placement. To guarantee intimate contact with BAG composition, the wound bed needs to be a uniform and flat surface.

(BAG) placement procedure: The procedure for implanting the device is analogous to the procedure used to implant Full thickness graft (entire epidermis and dermis are transplanted to the recipient site), and in this case said BAG composition is implanted proximally to a host tissue surface, placed with the mucous side facing outward and the remaining layer side facing inward in direct contact with the wound bed. Next, the graft is tacked into place by using interrupted sutures of 6-0 fast-absorbing catgut along its circumference and some sutures to affix the central part of the graft. The applicant prefers to use interrupted sutures rather than running sutures. The patient is placed under general anesthetic. Pain medications are utilized as necessary following surgery.

Dressings Fixation and compression are of significant importance to ensure the graft adheres to the wound without shear. The preferred Dressings to protect the BAG composition as follows: A: High-quality white nylon net. B: A Tubular Elastic Bandage applied over nylon net, to protect the site and to reduce the potential of shearing and graft dislodgement. The graft and the dressings can be left in place for extended periods without detrimental effects to the underlying wound.

Post-Operative details: To avoid unnecessary concerns, the patient should be made aware that a significant amount of serous and sero-sanguineous fluid may drain from the wound during the period of healing. Is normal that the graft changes color: of the white initial color to brown in the subsequent days. If a minor amount of bleeding occurs post-operatively or if the periphery of the graft losses viability, a slight crust or necrotic area can be gently removed.

Graft withdrawal: The applicant usually soaks the primary and secondary dressing with saline prior to removal the epidermal equivalent.

Second stage procedure: commonly the dermal analogue integrates with the patient's own cells and the neo-dermis exhibit a healthy granulation surface suitable for grafting a very thin autograft.

Complications: The following complications are possible with the use of any wound dressings. If any of the conditions occur, the BAG composition should be removed: infection, inflammation, allergic reaction, excessive redness, pain or swelling. Complications that the surgeon may encounter further than infection include seroma and/or hematoma formation, and graft contracture. Although wound infection is rare, when this is suspected, appropriate bacterial identification is obligatory and according to the cultivated microorganism's antibiotics should be prescribed. More extensive black necrotic tissue involving part or all of the grafted may signal partial or complete loss of the graft. Debridement should not be performed until the area of necrosis is clearly demarcated.

### EXAMPLE NO. 3

Determination of Humoral Immune Response in Graft Recipients

Forty patients 30 days after treatment were tested for a humoral immune response to a BAG graft component by mean to precipitation antibodies and skin test evaluation. The precipitating antibodies were tested by immunodiffusion in agar gel and skin test were done by intradermic application of 0.02 ml and evaluated at 10 minutes and at 48 hours.

The antigens were obtained by aqueous extracts in P.B.S. (Phosphate buffered saline) with pH 7.2.

The precipitating antibodies, the immediate and delayed skin test were negative in the 100% of the cases.

This study was conducted by the Dr. Roberto Maselli, Professor of Immunology of the Faculty of Medical Sciences (University of San Carlos of Guatemala) specialized in the University of Colorado, USA.

### INDUSTRIAL APPLICABILITY

This invention provides a proven technology to produce a non-immunogenic tissue graft compositions comprising esophagus mucosa, basement membrane and tunica submucosa of an esophagus of a warm-blooded vertebrate. The composition can be utilized for implanting at the site of tissue removal for immediate wound closure in cases where native tissue is damaged, in one embodiment, by trauma, or in another embodiment, by burns, cancer, vascular skin complications (chronic ulcers) or extensive injuries presenting in theaters of war (full-thickness burn-wounds defects).

The applicant observed that the composition has physical and physiological properties similar to that of the native tissue architecture of the organ to be replaced/repaired, since in a histological point of view is very similar to human skin, thus becomes an considerable importance of the invention and the general research intensity in the field of skin replacement therapy.

The patent aims to protect the claims related to the process for producing the composition, and claims relating to the methods used to implant the composition by surgical techniques in human subjects.

This application is being filed as a U.S. provisional patent application No. 61/583,138 filed 4 Jan. 2012 by the same applicant/inventor of the present application. The previous application claims have been reduced.

1. A Biologically active graft (BAG) composition capable of serving as a substitute for skin autograft utilized in skin replacement therapy, which has the effect of inducing stromal cells regeneration. It comprises esophagus mucosa, basement membrane and tunica submucosa as intact natural sheet forms (Ad integrum), delaminated from the tunica muscularis and adventitia of an esophagus of a warm-blooded vertebrate.

2. The Biologically active graft composition of claim 1, wherein the layers of that construct comprises non-keratinized stratified squamous epithelium (Mucosa), basement membrane and connective tissue (tunica submucosa) as intact natural sheet forms (Ad integrum) of an esophagus of a warm-blooded vertebrate.

3. The use of esophagus mucosa, basement membrane and tunica submucosa prepared without disruption of the natural cellular/nonliving structures from the esophagus of a warm-blooded vertebrate for the manufacture of a tissue graft construct capable of being utilized as a substitute for skin autograft in skin replacement therapy and for inducing stromal cells regeneration when implanted at a site in need of endogenous tissue growth.

4. A method for tissue grafting, comprising grafting a patient with a tissue graft of claim 1.

5. A method for inducing the formation of endogenous tissue at a site in need of endogenous tissue growth, said method consist of contacting said site with a Biologically Active Graft (BAG) composition of claim 1.
